(19) United States
(12) Patent Application Publication
(10) Pub. No.: US 2011/0243907 A1
(43) Pub. Date: Oct. 6, 2011

(54) COSMETIC AND NUTRITIONAL ALPHA-OMEGA COMPLEX COMPOSITION

(76) Inventor: Michael Anthony Nili, Reston, VA (US)

(21) Appl. No.: 13/047,715

(22) Filed: Mar. 14, 2011

Related U.S. Application Data
(60) Provisional application No. 61/321,107, filed on Apr. 5, 2010.

(51) Int. Cl. A61K 31/122; A61K 8/46; A61Q 19/00; A61Q 7/00; A61Q 5/00; A61Q 5/12; A61P 39/06; A61P 17/10

(52) U.S. Cl. 424/94.1; 514/678

(57) ABSTRACT

The present invention is an alpha omega complex composition for use in cosmetic and nutritional products and formulations for a user that contains coenzyme Q10, selenium sulfide and n-acetyl cysteine. Specific cosmetic and nutritional products and formulations include face cream, night cream, eye cream, lip cream, shampoos, conditioners, hand cream, body cream, daily dietary supplements, hair regeneration cream, anti-acne cream, anti-radiation cream, skin serum, eye-lash regeneration lotion and exfoliate products and formulations. One embodiment of the alpha omega complex composition would include 2.5% by volume NAC, 0.1% by volume Q10 and 0.05% by volume SeS₂.

This application claims priority to U.S. Provisional Application 61/321,107 filed on Apr. 5, 2010, the entire disclosure of which is incorporated by reference.

### TECHNICAL FIELD & BACKGROUND

The present invention generally relates to a cosmetic and nutritional alpha omega complex composition. More specifically, the invention is an alpha omega complex based composition that can be used in face cream, night cream, eye cream, lip cream, shampoos, conditioners, hand cream, body cream, daily dietary supplements, hair regeneration cream, anti-acne cream, anti-radiation cream, skin serum, eye-lash regeneration lotion and exfoliate products and formulations.

It is an object of the invention to provide an alpha omega complex based composition to promote human cell health and effectiveness.

It is an object of the invention to provide an alpha omega complex based composition to significantly improve cellular respiratory system activity by improving the regulation of the antioxidant mechanisms of cells.

What is really needed is a cosmetic and nutritional alpha omega complex based composition that can be conveniently taken to promote human cell health and effectiveness and significantly improve cellular respiratory system activity by improving the regulation of the antioxidant mechanisms of cells.

### DETAILED DESCRIPTION OF ILLUSTRATIVE EMBODIMENTS

Various aspects of the illustrative embodiments will be described using terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations are set forth in order to provide a thorough understanding of the illustrative embodiments. However, it will be apparent to one skilled in the art that the present invention may be practiced without the specific details. In other instances, well-known features are omitted or simplified in order not to obscure the illustrative embodiments.

Various operations will be described as multiple discrete operations, in turn, in a manner that is most helpful in understanding the present invention. However, the order of description should not be construed as to imply that these operations are necessarily order dependent. In particular, these operations need not be performed in the order of presentation.

The phrase “in one embodiment” is used repeatedly. The phrase generally does not refer to the same embodiment, however, it may. The terms “comprising”, “having” and “including” are synonymous, unless the context dictates otherwise.

The composition of the present invention is an alpha omega complex composition for use in cosmetic and nutritional products and formulations. The composition includes coenzyme Q10, selenium sulfide and n-acetyl cysteine and can include any combination of any amount of coenzyme Q10, selenium sulfide and n-acetyl cysteine. The alpha omega complex composition can be specifically used in cosmetic and nutritional products and formulations used in face cream, night cream, eye cream, lip cream, shampoos, conditioners, hand cream, body cream, daily dietary supplements, hair regeneration cream, anti-acne cream, anti-radiation cream, skin serum, eye-lash regeneration lotion and exfoliation products and formulations. The alpha omega complex composition can also be used in other cosmetic and nutritional products and formulations that are well known in the art as well. The alpha omega complex composition is the key active ingredient in these cosmetic and nutritional products and formulations. The alpha omega complex composition promotes cellular health and effectiveness and improves the user's cellular respiratory system by upregulating antioxidant mechanisms of the user's cells. Any amount of coenzyme Q10 can be combined with any amount of selenium sulfide and any amount of n-acetyle cysteine to form the alpha omega complex composition.

The alpha omega complex composition includes coenzyme Q10, also known as ubiquinone, ubidecarenone, coenzyme Q, and abbreviated at times as CoQ₁₀, CoQ, Q10, or simply Q, is a 1,4-benzoquinone, where Q refers to the quinone chemical group and 10 refers to the number of isoprenyl chemical subunits. The chemical structure of coenzyme Q10 is:

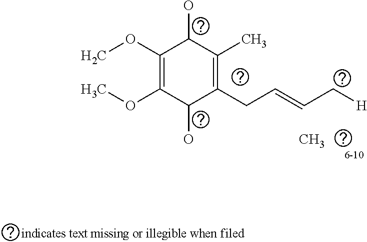

The molecular formula for coenzyme Q10 is C₅₉H₉₀O₄ and the chemical name for coenzyme Q10 is [2-(2E,6E,10E,14E,18E,22E,26E,30E,34E)-3,7,11,15,19,23,27,31,35,39-decamethyltetraconta-2,6,10,14,18,22,26,30,34,38-decaenyl]-5,6-dimethoxy-3-methylcyclohexa-2,5-diene-1,4-dione. Coenzyme Q10 is an oil-soluble substance present in most eukaryotic cells, primarily in the mitochondria. It is a component of the electron transport chain and participates in aerobic cellular respiration, generating energy in the form of adenosine-tri-phosphate (ATP). Ninety-five percent of the human body's energy is generated this way, therefore those organs with the highest energy requirements, such as the heart and the liver, have the highest CoQ₁₀ concentrations.

The alpha omega complex composition also includes selenium sulfide, also known as selenium (IV) sulfide, which is an inorganic compound with the approximate formula SeS₂. Both sulfur and selenium catenate (form chains and rings) readily and mixtures of selenium and sulfur likewise give rise to numerous sulfur and selenium compounds. The material commercially called selenium disulfide is sold as an antifungal agent in shampoos for the treatment of dandruff and seborrheic dermatitis associated in the scalp with Malassezia genus fungi. In the United States, a 1% strength is available over-the-counter and a 2.5% strength is also available with a prescription. At the 2.5% strength, selenium disulfide is also used on the body to treat tinea versicolor, a type of fungal skin infection caused by a different species of Malassezia.

Selenium sulfide has a composition that approximates SeS₂ and is sometimes called selenium disulfide. However as used in proprietary formulations, it is not a pure chemical compound but rather a mixture where the overall Se:S ratio is 1:2. The compounds are cyclic Se—S rings containing a variable number of S and Se atoms according to SeₙS₈₋ₙ. Selenium disulfide can cause discoloration of the hair and alter the color of hair dyes. It may also discolor metallic jewelry. Many selenium sulfides are well known. Selenium monosulfide (SeS) is the only selenium compound so far identified as a carcinogen in animals. Selenium monosulfide, along with elemental selenium and sulfur has been used, in past medicinal preparations, causing confusion and contradiction as to exactly what form selenium is in, in any given topical preparation. Below is a chemical structure for a selenium sulfide 1,2,3-Se₃S₅:

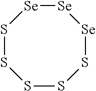

The alpha omega complex composition also includes n-acetyl cysteine, also known as acetylcysteine or N-acetyl-L-cysteine (abbreviated NAC), which is a pharmaceutical drug used mainly as a mucolytic agent and in the management of paracetamol (acetaminophen) overdoses. The chemical name for n-acetyle cysteine is (R)-2-acetamido-3-sulfanylpropanoic acid, with a molecular formula of C₅H₉NO₃S with the chemical structure of:

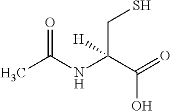

### Examples 1 and 2

One embodiment of the alpha omega complex composition would be 2.5% by volume NAC, 0.1% by volume Q10 and 0.05% by volume SeS₂. Another embodiment of the alpha omega complex composition includes a 1.0% by volume NAC, 0.1% by volume Q10 and 0.05% by volume SeS₂. Other suitable percentages by volume of NAC, Q10 and SeS₂ can also be incorporated into a formulation for the alpha omega complex composition.

While the present invention has been related in terms of the foregoing embodiments, those skilled in the art will recognize that the invention is not limited to the embodiments described. The present invention can be practiced with modification and alteration within the spirit and scope of the appended claims. Thus, the description is to be regarded as illustrative instead of restrictive on the present invention.

What is claimed is:

1. An alpha omega complex composition for use in cosmetic and nutritional products and formulations for a user, comprising:
coenzyme Q10;
selenium sulfide; and
n-acetyl cysteine.

2. The composition according to claim 1, wherein said coenzyme Q10 is 0.1% by volume.

3. The composition according to claim 1, wherein said selenium sulfide is 0.05% by volume and has a 1:2 Se:S ratio.

4. The composition according to claim 1, wherein said n-acetyl cysteine is 1.0% or 2.5% by volume.

5. The composition according to claim 1, wherein said cosmetic and nutritional products and formulations are face cream, night cream, eye cream, lip cream, shampoos, conditioners, hand cream, body cream, daily dietary supplements, hair regeneration cream, anti-acne cream, anti-radiation cream, skin serum, eye-lash regeneration lotion and exfoliate products and formulations.

6. The composition according to claim 1, wherein said composition is an active ingredient in said cosmetic and nutritional formulations.

7. An alpha omega complex composition for use in cosmetic and nutritional products and formulations for a user, comprising:
coenzyme Q10;
selenium sulfide; and
n-acetyl cysteine.

8. The composition according to claim 7, wherein said coenzyme Q10 is 0.1% by volume.

9. The composition according to claim 7, wherein said selenium sulfide is 0.05% by volume and has a 1:2 Se:S ratio.

10. The composition according to claim 7, wherein said n-acetyl cysteine is 1.0% or 2.5% by volume.

11. The composition according to claim 7, wherein said cosmetic and nutritional products and formulations are face cream, night cream, eye cream, lip cream, shampoos, conditioners, hand cream, body cream, daily dietary supplements, hair regeneration cream, anti-acne cream, anti-radiation cream, skin serum, eye-lash regeneration lotion and exfoliate products and formulations.

12. The composition according to claim 7, wherein said composition is an active ingredient in said cosmetic and nutritional formulations.

13. The composition according to claim 7, wherein said composition improves said user's cellular health and effectiveness.

14. An alpha omega complex composition for use in cosmetic and nutritional products and formulations for a user, comprising:
coenzyme Q10;
selenium sulfide; and
n-acetyl cysteine.

15. The composition according to claim 14, wherein said coenzyme Q10 is 0.1% by volume and said selenium sulfide is 0.05% by volume and has a 1:2 Se:S ratio.

16. The composition according to claim 14, wherein said n-acetyl cysteine is 1.0% or 2.5% by volume.

17. The composition according to claim 14, wherein said cosmetic and nutritional products and formulations are face cream, night cream, eye cream, lip cream, shampoos, conditioners, hand cream, body cream, daily dietary supplements, hair regeneration cream, anti-acne cream, anti-radiation cream, skin serum, eye-lash regeneration lotion and exfoliate products and formulations.

18. The composition according to claim 14, wherein said composition is an active ingredient in said cosmetic and nutritional formulations.

19. The composition according to claim 14, wherein said composition improves said user's cellular health and effectiveness.

20. The composition according to claim 14, wherein said composition improves said user's cellular respiratory system by upregulating antioxidant mechanisms of said user's cells.
